(19) United States
(12) Patent Application Publication
(10) Pub. No.: US 2013/0030364 A1
(43) Pub. Date: Jan. 31, 2013

(54) SINGLE USE SYRINGE WITH PLUNGER ARM SAFETY COVER

(71) Applicant: Ming-Yuan WU, Taipei City (TW)

(72) Inventor: Ming-Yuan WU, Taipei City (TW)

(21) Appl. No.: 13/191,864

(22) Filed: Jul. 27, 2011

(51) Int. Cl. A61M 5/50

(52) U.S. Cl. 604/110

(57) ABSTRACT

A single use syringe with plunger arm safety cover comprising a plunger arm, plunger tip, syringe body, and tip retaining sleeve is disclosed. The tip retaining sleeve surrounds tip retaining arms of the plunger tip allowing the tip retaining arms to grasp the plunger arm. After the plunger arm is pulled backwards, sleeve ribs prevent the tip retaining sleeve from moving in a forward direction. As the plunger arm is pushed forward, the tip retaining arms are no longer surrounded by the tip retaining sleeve and the tip retaining arms move outwards and release the plunger tip. After the plunger tip has been released from the plunger arm, the plunger tip and the plunger arm cannot be reconnected and the single use syringe cannot be reused. The plunger arm is removed from the syringe body and locked onto a guide lock and covers the needle tip.

### BACKGROUND OF THE INVENTION

1. Field of the Invention

The present invention relates to medical syringes. More specifically, the present invention discloses a safe and effective syringe that automatically ensures the syringe cannot be reused and provides a safety cover for the needle of the syringe using the plunger arm.

2. Description of the Prior Art

Syringes are commonly used medical implements for injecting medicine into a patient. A medical professional draws medicine into the syringe and then injects the medicine into a patient through a needle in the syringe.

While useful, the conventional syringe has various disadvantages that permit potentially dangerous use of the syringe. Misuse of the traditional syringe can be fatal or detrimental to a patient's health.

One major disadvantage of the conventional syringe is that the syringe can easily be used again. The conventional syringe or components of the syringe can easily be used again since the conventional syringe provides no inherent means to prevent reuse.

After a patient is injected with a medicine, residue such as blood, medicine, virus, bacteria, or other contaminants remain on the needle or inside the syringe. If another patient is injected using the used syringe, the second patient can be contaminated with residue from the first patient.

If the first patient is infected with a virus, the second patient is susceptible to contracting the virus from the previously used syringe. The second patient is also susceptible if the second patient is allergic to the medicine used with the first patient.

Additionally, it can be difficult to determine whether or not the syringe has been previously used.

Furthermore, exposed needles of the conventional syringe are potentially dangers to medical personnel.

Therefore, there is need for an improved medical syringe that effectively delivers medicine to a patient while automatically preventing reuse of the syringe.

### SUMMARY OF THE INVENTION

To achieve these and other advantages and in order to overcome the disadvantages of the conventional method in accordance with the purpose of the invention as embodied and broadly described herein, the present invention provides a syringe that automatically disconnects components of the syringe to prevent reuse and provides a safety cover over the needle after use.

The single use syringe of the present invention comprises a plunger arm, plunger tip, syringe body, and a tip retaining sleeve. The plunger arm comprises an arm post that is inserted into a mating hollow interior section of a tip base of the plunger tip. The tip retaining sleeve surrounds tip retaining arms of the plunger tip allowing the tip retaining arms to grasp the plunger tip.

When the plunger arm is pulled backwards, sleeve rings of the tip retaining sleeve press against the syringe body and the tip retaining sleeve is prevented from moving in a forward direction.

As the plunger arm is pushed forward, the tip retaining arms are no longer surrounded by the tip retaining sleeve and the tip retaining arms move outwards and release their grasp on the plunger arm. After the plunger tip has been released, the arm post of the plunger arm can continue to push the plunger tip forward until the end of the syringe body has been reached. However, after the plunger tip has been released, the plunger arm cannot grasp or pull the plunger tip backwards.

After the plunger tip has been released from the plunger arm, the plunger tip and the plunger arm cannot be reconnected and the single use syringe cannot be reused.

Features of the plunger arm and the syringe body cooperate to provide a retaining system that holds the plunger arm in position and prevents the plunger arm from moving backward or retracting during shipment or handling prior to use. When the single use syringe is put into use, the plunger arm is rotated and the retaining system is released allowing the plunger arm to be pulled backward.

After the single use syringe has been used the plunger arm is pulled backward and out of the syringe body. The plunger arm is then placed on a guide lock on the outside of the syringe body. As the plunger arm slides along the guide lock a channel in the plunger arm maintains the plunger arm in a proper alignment. When the tip of the plunger arm reaches the needle, the tip of the needle is moved into a gate opening of the channel. As the plunger arm continues forward the needle moves into the channel of the plunger arm and is trapped. The needle trapped in the front of channel prevents backward movement of the plunger arm. A lock tab on the plunger arm is then connected to the guide lock to hold the plunger arm to the syringe body. As a result, the needle is covered and trapped in the channel and the plunger arm is held in place on the guide lock of the syringe body. This provides an efficient and effective safety cover for the needle and prevents the needle from being exposed and potentially dangerous to medical personnel.

The plunger arm further comprises a used indicator. The used indicator comprises, for example, lettering or wording such as “USED” and/or a bright color. After the syringe has been used the used indicator allows easy confirmation that this syringe has been previously used. The used indicator is hidden and not visible until the syringe has been used.

Not only is the single use syringe of the present invention useful in preventing reuse of the syringe by medical professionals but also prevents addicts or intravenous drug users from sharing needles.

The single use syringe of the present invention dramatically decreases the need for needle exchange programs to ensure that intravenous drug users do not share needles. Even if the user does not return the used syringe to an exchange center, the single use syringe cannot be used by another addict.

These and other objectives of the present invention will become obvious to those of ordinary skill in the art after reading the following detailed description of preferred embodiments.

It is to be understood that both the foregoing general description and the following detailed description are exemplary, and are intended to provide further explanation of the invention as claimed.

### BRIEF DESCRIPTION OF THE DRAWINGS

The accompanying drawings are included to provide a further understanding of the invention, and are incorporated in and constitute a part of this specification.

The drawings illustrate embodiments of the invention and, together with the description, serve to explain the principles of the invention. In the drawings:

FIGS. 1A, 1B, and 1D are drawings illustrating side views of a plunger arm of a single use syringe with plunger arm safety cover according to an embodiment of the present invention;

FIG. 1C is a drawing illustrating a cross-sectional view of a plunger arm of a single use syringe with plunger arm safety cover according to an embodiment of the present invention;

FIG. 1E is a drawing illustrating an end view of a plunger arm of a single use syringe with plunger arm safety cover according to an embodiment of the present invention;

FIG. 1F is a drawing illustrating a side view of a knob of a plunger arm of a single use syringe with plunger arm safety cover according to an embodiment of the present invention;

FIG. 1G is a drawing illustrating a cross-sectional view of a knob of a plunger arm of a single use syringe with plunger arm safety cover according to an embodiment of the present invention;

FIG. 1H is a drawing illustrating an end view of a knob of a plunger arm of a single use syringe with plunger arm safety cover according to an embodiment of the present invention;

FIG. 2A is a drawing illustrating a side view of a tip retaining sleeve of a single use syringe with plunger arm safety cover according to an embodiment of the present invention;

FIG. 2B is a drawing illustrating a cross-sectional view of a tip retaining sleeve of a single use syringe with plunger arm safety cover according to an embodiment of the present invention;

FIGS. 3A-3B are drawings illustrating side views of a plunger tip of a single use syringe with plunger arm safety cover according to an embodiment of the present invention;

FIG. 3C is a drawing illustrating a cross-sectional view of a plunger tip of a single use syringe with plunger arm safety cover according to an embodiment of the present invention;

FIGS. 4A-4B are drawings illustrating side views of a syringe body of a single use syringe with plunger arm safety cover according to an embodiment of the present invention;

FIG. 4C is a drawing illustrating a cross-sectional view of a syringe body of a single use syringe with plunger arm safety cover according to an embodiment of the present invention;

FIG. 4D is a drawing illustrating an end view of a syringe body of a single use syringe with plunger arm safety cover according to an embodiment of the present invention;

FIG. 4E is a drawing illustrating a cross-sectional view of a sleeve stopper of a syringe body of a single use syringe with plunger arm safety cover according to an embodiment of the present invention;

FIG. 4F is a drawing illustrating a side view of a sleeve stopper of a syringe body of a single use syringe with plunger arm safety cover according to an embodiment of the present invention;

FIGS. 4G-4H are drawings illustrating end views of a sleeve stopper of a syringe body of a single use syringe with plunger arm safety cover according to an embodiment of the present invention;

FIGS. 5A-5B are drawings illustrating side views of a single use syringe with plunger arm safety cover according to an embodiment of the present invention;

FIG. 5C is a drawing illustrating a cross-sectional view of a single use syringe with plunger arm safety cover according to an embodiment of the present invention; and

FIGS. 6A-6G are drawings illustrating movement and operation of a single use syringe with plunger arm safety cover according to an embodiment of the present invention.

### DESCRIPTION OF THE PREFERRED EMBODIMENTS

Reference will now be made in detail to the preferred embodiments of the present invention, examples of which are illustrated in the accompanying drawings. Wherever possible, the same reference numbers are used in the drawings and the description to refer to the same or like parts.

The single use syringe with plunger arm safety cover of the present invention comprises a plunger assembly, a tip retaining sleeve, and a syringe body. The plunger assembly comprises a plunger arm and a plunger tip that removably attach together. The plunger assembly allows medicine to be drawn into the syringe body and the medicine to be injected into a patient.

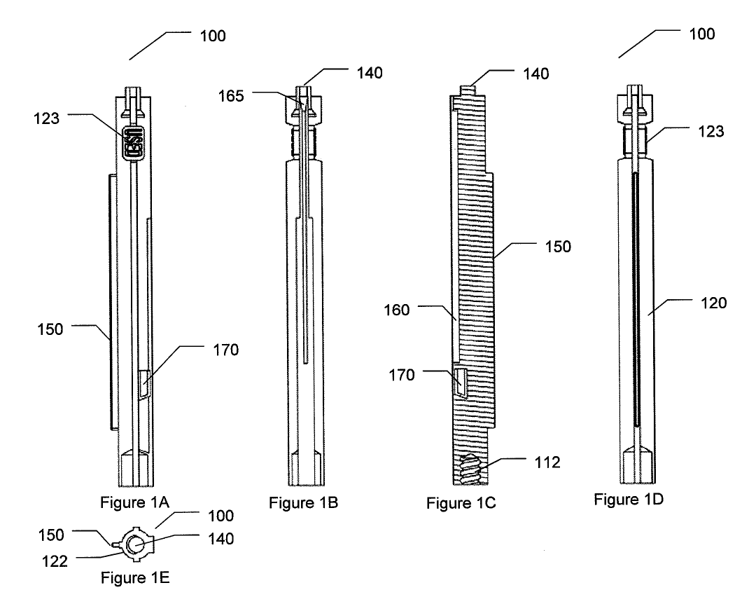

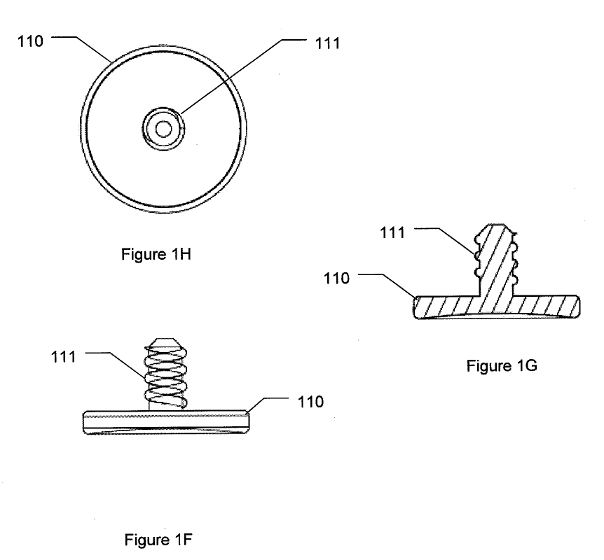

Refer to FIGS. 1A, 1B, and 1D, which are drawings illustrating side views of a plunger arm of a single use syringe with plunger arm safety cover according to an embodiment of the present invention and to FIG. 1C, which is a drawing illustrating a cross-sectional view of a plunger arm of a single use syringe with plunger arm safety cover according to an embodiment of the present invention and to FIG. 1E, which is a drawing illustrating an end view of a plunger arm of a single use syringe with plunger arm safety cover according to an embodiment of the present invention and to FIG. 1F, which is a drawing illustrating a side view of a knob of a plunger arm of a single use syringe with plunger arm safety cover according to an embodiment of the present invention and to FIG. 1G, which is a drawing illustrating a cross-sectional view of a knob of a plunger arm of a single use syringe with plunger arm safety cover according to an embodiment of the present invention and to FIG. 1H, which is a drawing illustrating an end view of a knob of a plunger arm of a single use syringe with plunger arm safety cover according to an embodiment of the present invention.

As shown in FIGS. 1A-1H the plunger arm 100 comprises a plunger knob 110, a plunger stem 120, tip flange 122, an arm post 140, a plunger catch 150, a channel 160, and a gate opening 165.

In an embodiment of the present invention the plunger knob 110 further comprises a threaded tip 111 that allows the plunger knob 110 to be screwed into stem threads 112 disposed in the end of the plunger stem 120.

In another embodiment of the present invention the plunger knob 110 is ultrasonically welded to the plunger stem 120.

The plunger knob 110 is positioned on one end of the plunger stem 120. The plunger knob 110 allows a medical professional or user of the syringe to pull back the plunger arm 100 of the plunger assembly in order to draw liquid into the syringe. The plunger knob 110 also allows a professional or user of the syringe to push on the plunger knob 110 in order to move the plunger assembly forward and expel liquid from the syringe.

Positioned on the other end of the plunger stem 120, the plunger arm 100 comprises at least one tip flange 122 and an arm post 140. The tip flange 122 and the arm post 140 cooperate with the plunger tip to position and hold the plunger tip of the plunger assembly.

The plunger stem further comprises a used indicator 123. The used indicator 323 comprises, for example, lettering or wording such as “USED” and/or a bright color. After the syringe has been used the used indicator 123 allows a user to easily identify that this syringe has been previously used. The used indicator 123 is hidden and not visible until the syringe has been used.

The plunger catch 150 and the syringe body cooperate to provide a retaining system that holds the plunger arm 100 in position and prevents the plunger arm 100 from moving backward or retracting during shipment or handling prior to use. When the single use syringe is put into use, the plunger arm 100 is rotated and the plunger catch 150 enters a key-way in the syringe body allowing the plunger arm 100 to be pulled backward.

The plunger stem 120 further comprises a lock tab 170. After the plunger arm 100 has been removed from the syringe body and attached to the guide lock, the lock tab 170 connects to the guide lock to hold the plunger arm 100 to the syringe body.

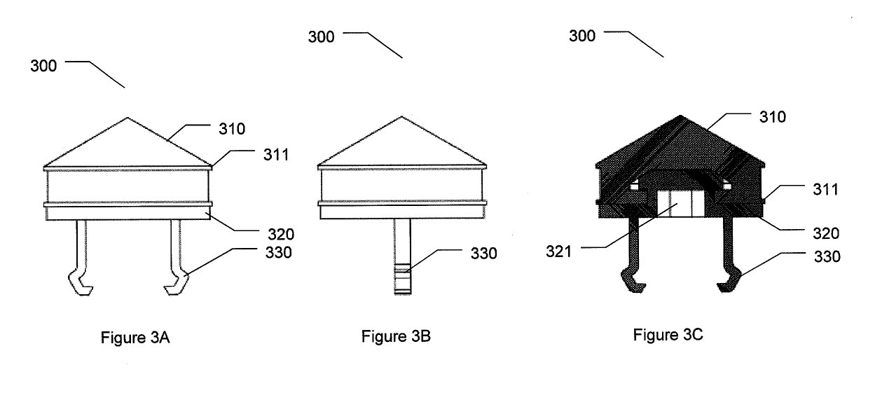

Refer to FIGS. 3A-3B, which are drawings illustrating side views of a plunger tip of a single use syringe with plunger arm safety cover according to an embodiment of the present invention and to FIG. 3C, which is a drawing illustrating a cross-sectional view of a plunger tip of a single use syringe with plunger arm safety cover according to an embodiment of the present invention. The plunger assembly of the single use syringe with plunger arm safety cover of the present invention further comprises a plunger tip 300 that connects with the plunger arm.

The plunger tip 300 comprises a tip head 310 and a tip base 320. The tip base 320 comprises a base opening 321 that mates with the arm post (140 FIG. 1A). The tip base 320 further comprises tip retaining arms 330 to grasp the tip flange (122 FIG. 1E) and hold the plunger tip 300 to the plunger arm.

In an embodiment of the present invention the base opening 321 and the arm post are formed in a mating non-round shape to prevent the plunger tip 300 from rotating separately from the plunger arm. For example, the arm post and the base opening 321 are square, triangle, or diamond shaped. As a result, when the arm post is inserted into the base opening 321 the plunger tip 300 is only allowed to rotate together with the plunger arm.

The tip head 310 further comprises tip head rings 311 that improve the seal between the tip head 310 and the syringe body and the suction characteristics of the single use syringe.

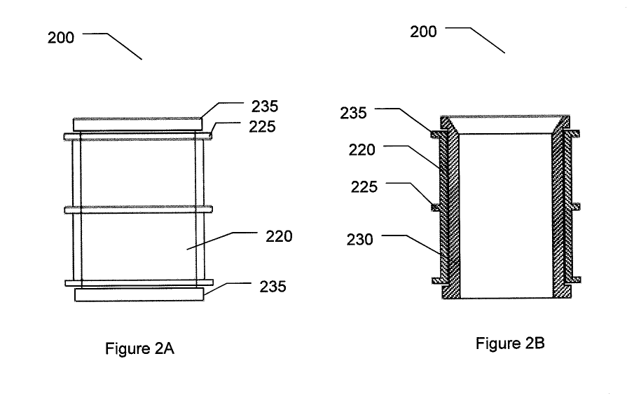

Refer to FIG. 2A, which is a drawing illustrating a side view of a tip retaining sleeve of a single use syringe with plunger arm safety cover according to an embodiment of the present invention and to FIG. 2B, which is a drawing illustrating a cross-sectional view of a tip retaining sleeve of a single use syringe with plunger arm safety cover according to an embodiment of the present invention.

The single use syringe with plunger arm safety cover of the present invention further comprises a tip retaining sleeve 200. The tip retaining sleeve 200 is an elongated tube that is positioned around the plunger arm. A plurality of sleeve ribs 225 are disposed on the tip retaining sleeve 200 and extend outside of the main body of the tip retaining sleeve 200.

In an embodiment of the present invention the inside surface of the tip retaining sleeve 200 is angled or has a chamfered end to allow the tip retaining arms to exit the tip retaining sleeve more easily and release the plunger tip sooner.

In an embodiment of the present invention the tip retaining sleeve 200 comprises an inner plastic sleeve 230 and an outer rubber sleeve 220. The plastic sleeve 230 is disposed inside the rubber sleeve 220. Sleeve tabs 235 on the ends of the plastic sleeve 230 hold the rubber sleeve 220 to the plastic sleeve 230 and prevent the rubber sleeve 220 from sliding off of the plastic sleeve 230.

The inner plastic sleeve 230 reduces friction between the tip retaining sleeve 200 and the plunger arm. The sleeve ribs 225 of the outer rubber sleeve 220 cooperate with the syringe body to prevent forward movement of the tip retaining sleeve 200.

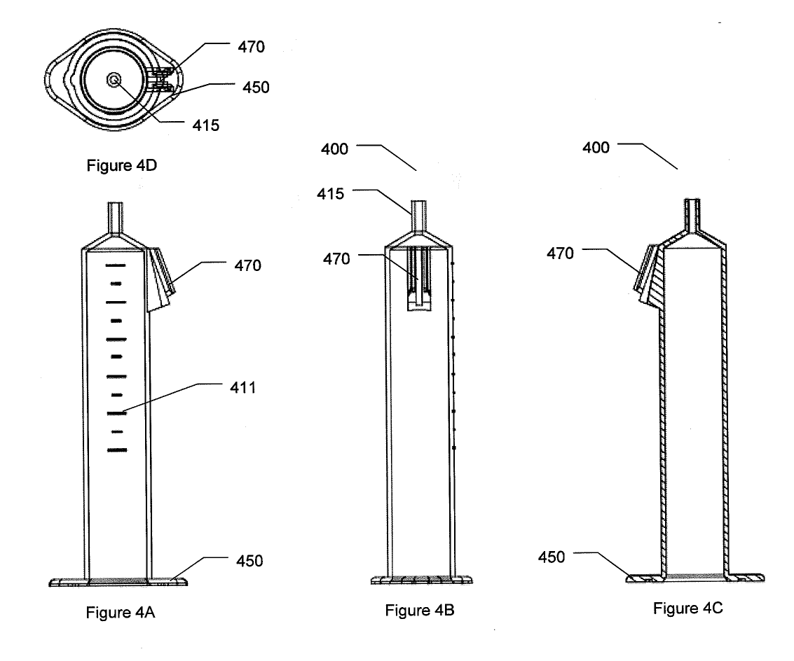

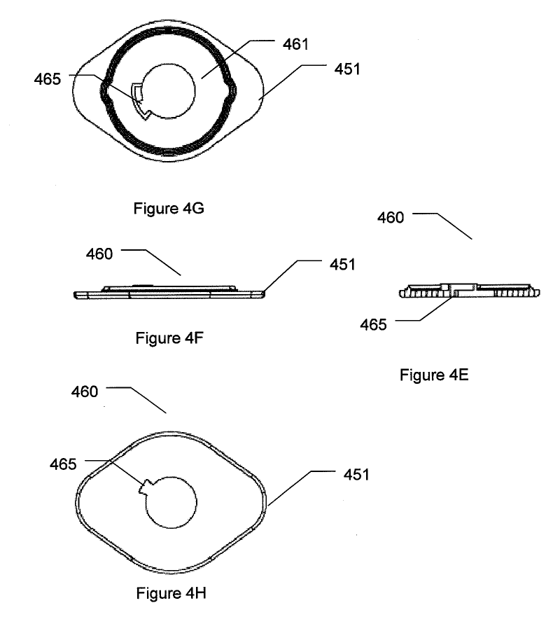

Refer to FIGS. 4A-4B, which are drawings illustrating side views of a syringe body of a single use syringe with plunger arm safety cover according to an embodiment of the present invention and to FIG. 4C, which is a drawing illustrating a cross-sectional view of a syringe body of a single use syringe with plunger arm safety cover according to an embodiment of the present invention and to FIG. 4D, which is a drawing illustrating an end view of a syringe body of a single use syringe with plunger arm safety cover according to an embodiment of the present invention and to FIG. 4E, which is a drawing illustrating a cross-sectional view of a sleeve stopper of a syringe body of a single use syringe with plunger arm safety cover according to an embodiment of the present invention and to FIG. 4F, which is a drawing illustrating a side view of a sleeve stopper of a syringe body of a single use syringe with plunger arm safety cover according to an embodiment of the present invention and to FIGS. 4G-4H, which are drawings illustrating end views of a sleeve stopper of a syringe body of a single use syringe with plunger arm safety cover according to an embodiment of the present invention.

The single use syringe of the present invention further comprises a syringe body 400. The syringe body 400 comprises a syringe body tip 415, a guide lock 470, a body grasp flange 450, a sleeve stopper 460, and a key-way 465.

A needle for injecting a patient is molded into the syringe body tip 415.

The body grasp flange 450 extends outwardly from the syringe body 400 to assist a professional or user during operation of the syringe. To aid in pulling back the plunger assembly the syringe user places a finger or fingers on the top side of the body grasp flange 450 while pulling back on the plunger knob. When pressing on the plunger knob to move the plunger assembly forward the syringe user places a finger or fingers on the bottom side of the body grasp flange 450.

The sleeve stopper 460 comprises a second body grasp flange 451 that mates with the first body grasp flange 450 and a sleeve stopper wall 461 that prevents the tip retaining sleeve (200 FIGS. 2A 2B) from exiting the syringe body 400.

The sleeve stopper 460 further comprises a key-way 465. The sleeve stopper 460 and the sleeve stopper wall 461 cooperate with the plunger catch (150 FIG. 1A) to prevent movement of the plunger arm until the plunger catch aligns with the key-way 465. Only when the plunger catch is aligned with the key-way 465, the plunger arm is allowed to be pulled backward and the single use syringe can be operated.

The syringe body 100 further comprises volume markers 411 that allow indication of the volume of medicine or liquid contained in the syringe body 100.

After the single use syringe has been used the plunger arm is pulled backward and out of the syringe body 400. The plunger arm is then placed on the guide lock 470 on the outside of the syringe body 400.

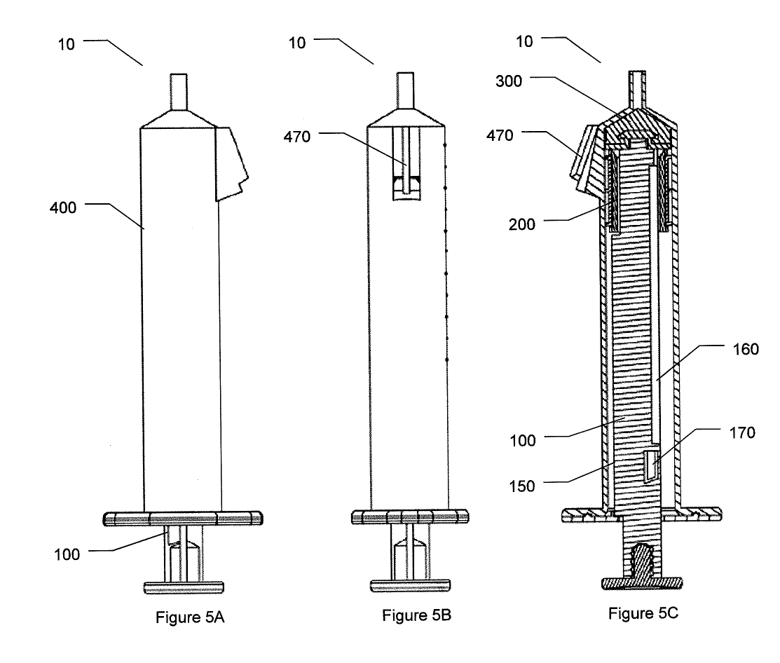

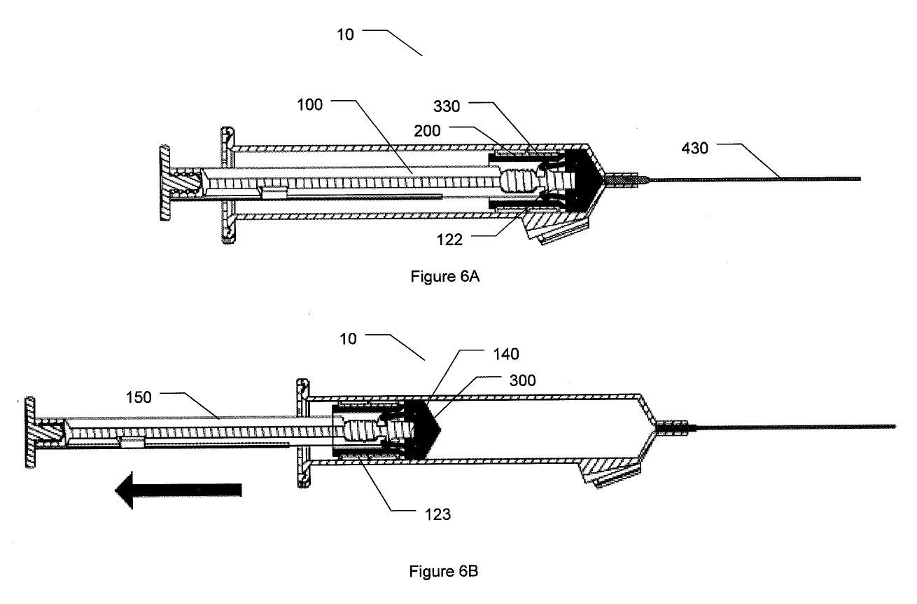

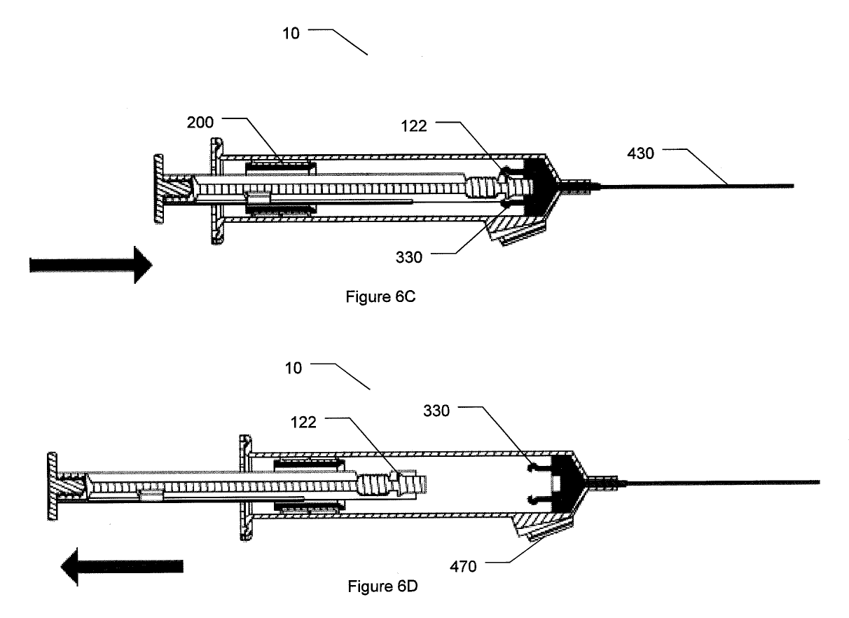

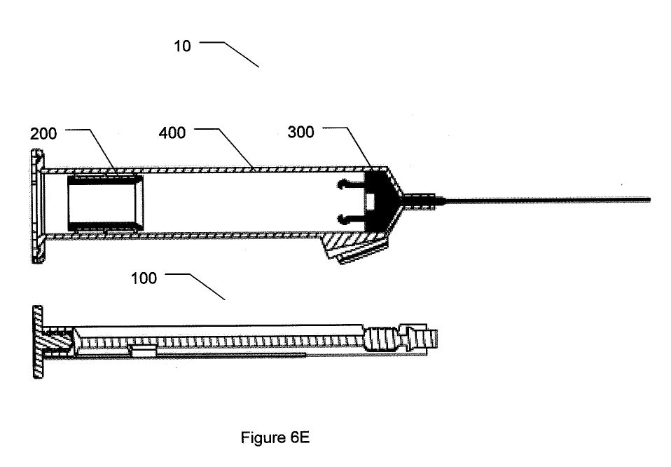

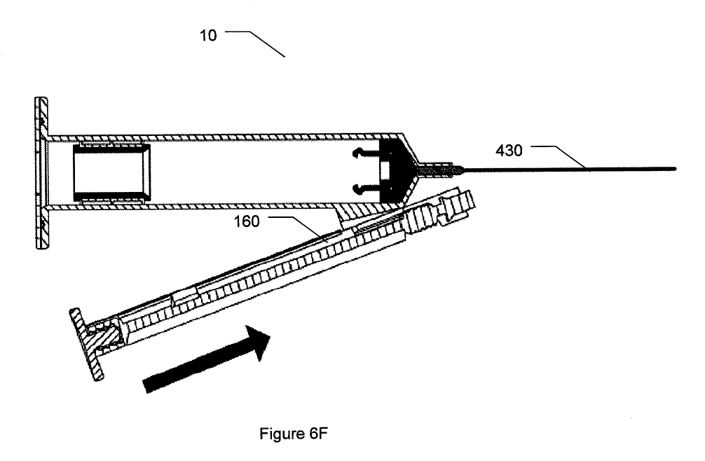

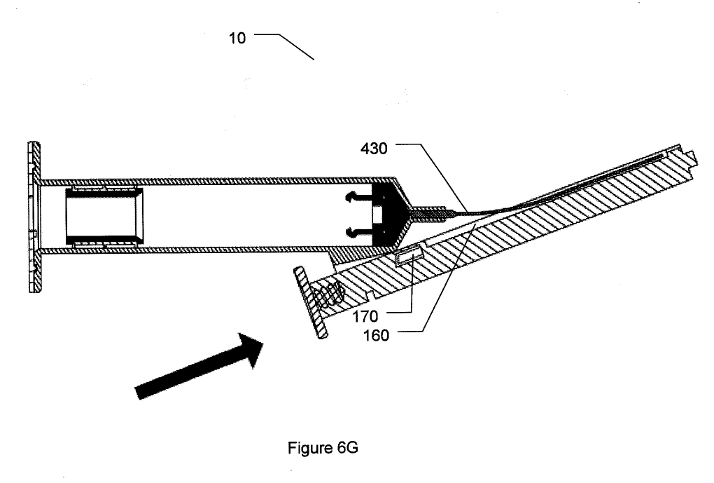

Refer to FIGS. 5A-5B, which are drawings illustrating side views of a single use syringe with plunger arm safety cover according to an embodiment of the present invention and to FIG. 5C, which is a drawing illustrating a cross-sectional view of a single use syringe with plunger arm safety cover according to an embodiment of the present invention and to FIGS. 6A-6G, which are drawings illustrating movement and operation of a single use syringe with plunger arm safety cover according to an embodiment of the present invention and continue to refer to FIGS. 1A-4H.

Prior to use, the elements of the single use syringe with plunger arm safety cover 10 of the present invention are positioned as shown in FIG. 5C. The plunger tip 300 is positioned at the forward area of the interior of the syringe body 400. The arm post 140 of the plunger arm 100 is positioned inside the base opening 321 of the tip base 320 of the plunger tip 300. The tip retaining sleeve 200 surrounds the tip retaining arms 330 of the plunger tip 300 and holds the tip retaining arms 330 grasping the tip flange 122 to hold the plunger tip 300 and the plunger arm 100 together.

The plunger catch 150 is trapped against the sleeve stopper wall 461 and prevents the plunger arm 100 from moving backward in the syringe body 400. While the rear end of the plunger catch 150 is trapped by the sleeve stopper wall 461 the front end of the plunger catch 150 prevents the tip retaining sleeve 200 from moving backward.

After the single use syringe 10 has been used the plunger arm 100 is pulled backward and out of the syringe body 400. The plunger arm 100 is then placed on the guide lock 470 on the outside of the syringe body 400.

As the plunger arm 100 slides along the guide lock 470 the channel 160 in the plunger arm 100 maintains the plunger arm 100 in a proper alignment. When the tip of the plunger arm 100 reaches the needle 430, the tip of the needle 430 is moved into a gate opening 165 of the channel 160. As the plunger arm 100 continues forward the needle 430 moves into the channel 160 of the plunger arm 100 and is trapped and bends. The lock tab 170 is then connected to the guide lock 470 to hold the plunger arm 100 to the syringe body 400. The lock tab 170 and the guide lock 470 comprise a mating configuration that allows the lock tab 170 and the guide lock 470 to attach together to hold the plunger arm 100 to the syringe body 400 and prevent the plunger arm 100 from moving while covering the needle 430 and while the needle 430 is trapped in the channel 160.

When assembled, the single use syringe 10 of the present invention comprises the plunger assembly (plunger arm 100 and plunger tip 300) and the tip retaining sleeve 200 disposed inside the syringe body 400 as previously described regarding FIG. 5C.

As the plunger arm 100 is pulled back the tip retaining sleeve 200 holds the tip retaining arms 330 in to grasp the tip flange 122 so that the plunger tip 300 is pulled back along with the plunger arm 100. As the plunger tip 300 is pulled back the plunger head 310 pulls the tip retaining sleeve 200 back with the plunger tip 300.

As the tip retaining sleeve 200 moves backwards in the syringe body 400, the sleeve ribs 225 of the tip retaining sleeve 200 contact the interior wall of the syringe body 400. At any time after backward movement, the friction between the sleeve ribs 225 and the interior wall of the syringe body 400 prevents the tip retaining sleeve 200 from moving in the forward direction.

As the plunger arm 100 continues to be pulled backward the tip retaining sleeve 200 continues to move backwards along with the plunger assembly (plunger arm 100 and plunger tip 300).

When the tip retaining sleeve 200 reaches the rear opening of the syringe body 400, the sleeve stopper 460 of the syringe body 400 blocks the backward movement of the tip retaining sleeve 200 and prevents the tip retaining sleeve 200 from exiting the syringe body 400. At this position the tip retaining sleeve 200 is prevented from moving backward by the sleeve stopper 460 and prevented from moving forward by the sleeve ribs 225 pressing against the interior wall of the syringe body 400.

When the plunger knob 110 is pushed the plunger arm 100 moves forward. Since the tip retaining sleeve 200 is prevented from moving forward, the tip retaining arms 330 move outwards as the tip retaining sleeve 200 no longer surrounds the tip retaining arms 330. After the tip retaining arms 330 have moved outwards and away from the plunger arm 100, the tip retaining arms 330 no longer grasp the tip flange 122 of the plunger arm 100. However, the plunger tip 300 can continue to be moved in a forward direction since it is now pushed by the arm post 140.

When the plunger assembly has been moved all the way forward, the forward end of the syringe body 400 stops forward movement of the plunger tip 300.

When the plunger arm 100 is now pulled backwards the plunger tip 300 is separated from the plunger arm 100 since the tip retaining arms 330 no longer grasp the tip flange 122 of the plunger arm 100.

After the plunger tip 300 has been released from the plunger arm 100, the plunger arm 100 cannot reconnect with the plunger tip 300 and the single use syringe 10 cannot be used again.

At any time after the tip retaining arms 330 are not surrounded by the tip retaining sleeve 200 and have moved outwards, the plunger tip 300 has been released from the plunger arm 100 and the plunger tip 300 can no longer be pulled backwards by the plunger arm 100.

After the plunger arm 100 has moved forward the used indicator 123 of the plunger arm 100 provides visual identification and confirmation that the syringe has been used.

Utilizing a plurality of sleeve ribs 225 allows different dosage amounts with a single sized syringe while preventing reuse of the syringe. For example, if the dose requires the plunger assembly to travel backwards 25% of the interior of the syringe body, the sleeve ribs 225 prevent the tip retaining sleeve 200 from moving forward and the plunger tip 300 is released from the plunger arm 100. If the dose requires the plunger assembly to travel backwards 50% of the interior of the syringe body, the sleeve ribs 225 prevent the tip retaining sleeve 200 from moving forward and the plunger tip 300 is released from the plunger arm 100.

After the single use syringe 10 has been used the plunger arm 100 is pulled backward and out of the syringe body 400. The plunger arm 100 is then placed on the guide lock 470 on the outside of the syringe body 400.

As the plunger arm 100 slides along the guide lock 470 the channel 160 in the plunger arm 100 maintains the plunger arm 100 in a proper alignment. When the tip of the plunger arm 100 reaches the needle 430, the tip of the needle 430 is moved into a gate opening 165 of the channel 160. As the plunger arm 100 continues forward the needle 430 moves into the channel 160 of the plunger arm 100 and is trapped and bends. The lock tab 170 is then connected to the guide lock 470 to hold the plunger arm 100 to the syringe body 400. The lock tab 170 and the guide lock 470 comprise a mating configuration that allows the lock tab 170 and the guide lock 470 to attach together to hold the plunger arm 100 to the syringe body 400 and prevent the plunger arm 100 from moving while covering the needle 430 and while the needle 430 is trapped in the channel 160. As a result, the needle 430 is covered and trapped in the channel 160 and the plunger arm 100 is held in place on the guide lock 470 of the syringe body 400. This provides an efficient and effective safety cover for the needle 430 and prevents the needle 430 from being exposed and potentially dangerous to medical personnel.

Additionally, when the needle 430 is covered by the plunger arm 100 the used indicator 123 of the plunger arm 100 is clearly visible and alerts medical personnel that this syringe has been used.

In an embodiment of the present invention the tip retaining arms are disposed on the plunger arm and the tip flange is disposed on the plunger arm plunger tip. When the tip retaining sleeve surrounds the tip retaining arms of the plunger arm, the tip retaining arms grasp the tip flange of the plunger tip and the plunger tip is held to the plunger arm. When the plunger arm moves forward the tip retaining arms of the plunger arm move outwards and the plunger tip is released from the plunger arm.

It will be apparent to those skilled in the art that various modifications and variations can be made to the present invention without departing from the scope or spirit of the invention. In view of the foregoing, it is intended that the present invention cover modifications and variations of this invention provided they fall within the scope of the invention and its equivalent.

What is claimed is:

1. A syringe comprising:
a plunger tip, the plunger tip comprising at least one tip retaining arm;
a plunger arm, the plunger arm comprising:
an arm post for inserting into a base opening of the plunger tip;
a tip flange for allowing the at least one tip retaining arm to grasp the plunger arm; and
a channel for covering a needle of the syringe after use;
a tip retaining sleeve surrounding the at least one tip retaining arm while the at least one tip retaining arm is grasping the tip flange; and
a syringe body holding the plunger arm, the plunger tip, and the tip retaining sleeve, the syringe body comprising a guide lock for holding the plunger arm to the syringe body after the syringe has been used.

2. The syringe of claim 1, the tip retaining sleeve comprising at least one sleeve ring for impeding forward movement of the tip retaining sleeve.

3. The syringe of claim 1, the plunger arm further comprising:
a tab lock for mating with the guide lock to hold the plunger arm to the syringe body.

4. The syringe of claim 1, further comprising:
a catch disposed on the plunger arm; and
a key-way disposed on the syringe body, the plunger arm only allowed backward movement when the catch is aligned with the key-way.

5. The syringe of claim 1, the syringe body further comprising:
a sleeve stopper for preventing the tip retaining sleeve from exiting the syringe body.

6. The syringe of claim 1, the tip retaining sleeve comprising:
an inner plastic sleeve; and
an outer rubber sleeve.

7. The syringe of claim 1, the channel further comprising:
a gate opening for guiding the needle into the channel.

8. The syringe of claim 1, wherein after the syringe has been used the plunger arm is removed from the syringe body, the plunger arm is positioned with the channel on the guide lock, the plunger arm is pushed forward until the needle enters the channel, and the plunger arm is held to the syringe body by the guide lock.

9. A syringe comprising:
a plunger tip, the plunger tip comprising at least one tip retaining arm;
a plunger arm, the plunger arm comprising:
an arm post for inserting into a base opening of the plunger tip;
a tip flange for allowing the at least one tip retaining arm to grasp the plunger arm; and
a channel for covering a needle of the syringe after use;
a tip retaining sleeve surrounding the at least one tip retaining arm while the at least one tip retaining arm is grasping the tip flange, the tip retaining sleeve comprising at least one sleeve ring for impeding forward movement of the tip retaining sleeve; and
a syringe body holding the plunger arm, the plunger tip, and the tip retaining sleeve, the syringe body comprising a guide lock disposed on an exterior surface of the syringe body for holding the plunger arm to the syringe body after the syringe has been used.

10. The syringe of claim 9, the plunger arm further comprising:
a tab lock for mating with the guide lock to hold the plunger arm to the syringe body.

11. The syringe of claim 9, further comprising:
a catch disposed on the plunger arm; and
a key-way disposed on the syringe body, the plunger arm only allowed backward movement when the catch is aligned with the key-way.

12. The syringe of claim 9, the syringe body further comprising:
a sleeve stopper for preventing the tip retaining sleeve from exiting the syringe body.

13. The syringe of claim 9, the tip retaining sleeve comprising:
an inner plastic sleeve; and
an outer rubber sleeve.

14. The syringe of claim 9, the channel further comprising:
a gate opening for guiding the needle into the channel.

15. The syringe of claim 9, wherein after the syringe has been used the plunger arm is removed from the syringe body, the plunger arm is positioned with the channel on the guide lock, the plunger arm is pushed forward until the needle enters the channel, and the plunger arm is held to the syringe body by the guide lock.

16. A syringe comprising:
a plunger tip, the plunger tip comprising at least one tip retaining arm;
a plunger arm, the plunger arm comprising:
an arm post for inserting into a base opening of the plunger tip;
a tip flange for allowing the at least one tip retaining arm to grasp the plunger arm;
a channel for covering a needle of the syringe after use;
a catch; and
a tab lock;
a tip retaining sleeve surrounding the at least one tip retaining arm while the at least one tip retaining arm is grasping the tip flange, the tip retaining sleeve comprising at least one sleeve ring for impeding forward movement of the tip retaining sleeve; and
a syringe body holding the plunger arm, the plunger tip, and the tip retaining sleeve, the syringe body comprising:
a sleeve stopper for preventing the tip retaining sleeve from exiting the syringe body;
a guide lock disposed on an exterior surface of the syringe body, the tab lock of the plunger arm and the guide lock connecting to hold the plunger arm to the syringe body after the syringe has been used; and
a key-way disposed on the syringe body, the plunger arm only allowed backward movement when the catch on the plunger arm is aligned with the key-way.

17. The syringe of claim 16, where the at least one tip retaining arm releases grasp of the plunger arm after exiting the tip retaining sleeve.

18. The syringe of claim 16, the tip retaining sleeve comprising:
an inner plastic sleeve; and
an outer rubber sleeve.

19. The syringe of claim 16, the channel further comprising:
a gate opening for guiding the needle into the channel.

20. The syringe of claim 16, wherein after the syringe has been used the plunger arm is removed from the syringe body, the plunger arm is positioned with the channel on the guide lock, the plunger arm is pushed forward until the needle enters the channel, and the plunger arm is held to the syringe body by the guide lock.
